(19) United States
(12) Patent Application Publication
(10) Pub. No.: US 2007/0292842 A1
(43) Pub. Date: Dec. 20, 2007

(54) Detection of viral or viral vector integration sites in genomic DNA

(76) Inventors: Stephanie B. Fulmer-Smentek, Cupertino, CA (US); Douglas N. Roberts, Campbell, CA (US); Douglas A. Amorese, Los Altos, CA (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 11/453,205

(22) Filed: Jun. 14, 2006

(51) Int. Cl. C12Q 1/70; C12Q 1/68; C12N 15/86

(52) U.S. Cl. 435/5; 435/6; 435/456

(57) ABSTRACT

Methods for detecting the integration of viral nucleic acids into a host cell, and methods for determining the locus of integration using microarrays are described. The methods can also be used in conjunction with viral vectors used in gene therapy.

### BACKGROUND

Gene therapy using viral vectors is a promising technique for treating certain diseases, and for improving therapy outcomes for certain diseases. For example, retrovirus-mediated stem cell therapy is currently being used to treat nonmalignant diseases, such as leukemia. Similarly, adeno-associated viruses are being developed as delivery vectors for gene therapy, because of their nonpathogenic and nonimmunogenic properties.

Viral vectors are usually inactivated so that they are incapable of integration and therefore incapable of infecting the host organism. When retroviral or adeno-associated viral constructs are used as gene therapy vectors, however, there is concern that the virus will become integrated into the host cell (i.e. human) genome. This risk is because these viral constructs can cause infection in their wild-type state, either by recombination, or by targeted integration. These integration events can have deleterious effects on the gene therapy patient. Knowledge of integration events and determination of the location of integration in the host cell genome is therefore critical.

Current methods for studying viral integration involve techniques such as Southern blotting, where genomic DNA is harvested, blotted and then detected using a labeled DNA probe. This method can detect the presence of a virus, but provides no information about the location of integration. Cloning methods have also been used, where pieces of genomic DNA containing the virus are cloned and then sequenced to determine the sequence surrounding the integration site. Such methods are labor intensive and may not detect many secondary integration events.

### SUMMARY

This patent is directed to methods and devices for detecting viral nucleic acids. Embodiments include detecting the presence of a viral vector in a host cell, detecting integration of viral nucleic acids, etc.

In embodiments, the methods described herein comprise generating nucleic acid fragments from a host cell genome and hybridizing these fragments to a microarray. A second set of nucleic acid fragments is used as a probe to detect the viral nucleic acid fragments on the microarray. The location of the detected fragments provides information on the site of integration.

Another aspect provides DNA arrays that can be used to identify viral nucleic acids or viral vectors in a host cell, or the location on the genome where a virus would integrate. In an embodiment, the arrays contain probe sequences complementary to host cell genomic DNA, with the probes laid down at regular intervals along the length of the genome. The arrays can be used to detect the presence of a viral vector and can also be used to determine the location of integration of a virus into the host cell genome.

In another aspect, kits that include arrays and compositions for identifying or detecting viral nucleic acids in a host cell are provided. The kits include one or more arrays containing probe sequences to genomic DNA, along with reagents necessary for amplification and labeling.

### BRIEF DESCRIPTION OF THE DRAWINGS

FIG. 1 shows an exemplary substrate carrying an array, such as may be used in the devices described herein.

FIG. 2 shows an enlarged view of a portion of FIG. 1 showing spots or features.

FIG. 3 is an enlarged view of a portion of the substrate of FIG. 1.

FIG. 4 shows a graphical illustration of a method for generating and amplifying nucleic acid fragments from a host cell.

FIG. 5 shows a graphical illustration of a method used to identify a viral nucleic acid after the viral nucleic acid is integrated into the host cell genome, and to determine the site of the integration in the host genome.

### DETAILED DESCRIPTION

Various embodiments will be described in detail with reference to the drawings, wherein like reference numerals represent like parts throughout the several views. Reference to various embodiments does not limit the scope of the claims attached hereto. Additionally, any examples set forth in this specification are not intended to be limiting and merely set forth some of the many possible embodiments for the claims.

Unless defined otherwise, all technical and scientific terms used herein have the same meaning as commonly understood to one of ordinary skill in the art. Although any methods, devices and material similar or equivalent to those described herein can be used in the practice or testing of the methods herein, the methods, devices and materials are now described.

All publications and patent applications in this specification are indicative of the level of ordinary skill in the art and are incorporated herein by reference in their entireties.

The term “genome” refers to all nucleic acid sequences (coding and non-coding) and elements present in or originating from a single cell or each cell type in an organism. The term genome also applies to any naturally occurring or induced variation of these sequences that may be present in a normal, mutant or disease variant of any virus or cell type. These sequences include, but are not limited to, those involved in the maintenance, replication, segregation, and higher order structures (e.g. folding and compaction of DNA in chromatin and chromosomes), or other functions, if any, of the nucleic acids as well as all the coding regions and their corresponding regulatory elements needed to produce and maintain each particle, cell or cell type in a given organism. For example, the human genome consists of approximately 3×10⁹ base pairs of DNA organized into distinct chromosomes. The genome of a normal diploid somatic human cell consists of 22 pairs of autosomes (chromosomes 1 to 22) and either chromosomes X and Y (males) or a pair of X chromosomes (female) for a total of 46 chromosomes. A genome of a cancer cell may contain variable numbers of each chromosome in addition to deletions, rearrangements and amplification of any subchromosomal region or DNA sequence.

The term “nucleic acid” as used herein means a polymer composed of nucleotides, e.g., deoxyribonucleotides or ribonucleotides, or compounds produced synthetically (e.g., PNA as described in U.S. Pat. No. 5,948,902 and the references cited therein) which can hybridize with naturally occurring nucleic acids in a sequence specific manner analogous to that of two naturally occurring nucleic acids, e.g., can participate in Watson-Crick base pairing interactions.

The terms “ribonucleic acid” and “RNA” as used herein mean a polymer composed of ribonucleotides.

The terms “deoxyribonucleic acid” and “DNA” as used herein mean a polymer composed of deoxyribonucleotides.

A “host cell” is a cell that has been infected with a virus or other microorganism. Viruses use host cells as a part of their life cycles, using the processes of the host cell to reproduce themselves. The host cells include, but are not limited to, eukaryotic cells, mammalian cells, etc.

The term “virus” refers to a submicroscopic parasite capable of infecting a host cell. Typically, viruses carry a small amount of genetic material, in the form of viral nucleic acids (either DNA or RNA), encapsulated by a protective coating consisting of proteins, lipids, glycoproteins, or a combination of proteins, lipids and glycoproteins. For the purposes of this description, the terms “virus” and “viral nucleic acid” are used interchangeably. Some viruses (such as retroviruses, for example) can only replicate by integrating into the host cell genome, while others (such as adeno-associated viruses (AAV) can replicate without integration.

A “viral vector” is a viral nucleic acid construct used experimentally or in gene therapy. Commonly used gene therapy viral vectors include adeno-associated viral vectors or recombinant adeno-associated viral vectors. Viral gene therapy vectors are altered to be replication-deficient, such that integration is not possible and the viral vector cannot cause disease. However, wild-type (i.e. unaltered) viruses and viral vectors can integrate into the genome, and when used in gene therapy, can cause deleterious effects, such as oncogene activation, knocking out tumor suppressor genes, etc.

The term “provirus” refers to a virus that has integrated itself into the host cell. The term “proviral DNA” refers to the DNA of a virus that is inserted into the host cell genome in an infected cell. The terms “provirus” and “proviral DNA” are used interchangeably herein

The term “retrovirus” refers to a member of a class of viruses that have their genetic material in the form of RNA and use the reverse transcriptase enzyme to translate their RNA into DNA in the host cell.

The term “sample” as used herein relates to a material or mixture of materials, typically, although not necessarily, in fluid form, containing one or more components of interest. Samples include, but are not limited to, biological fluid samples containing eukaryotic or mammalian host cells, and include host cells derived from gene therapy patients, for example. Samples may also be derived from natural biological sources such as cells or tissues. A “biological fluid” includes, but is not limited to, blood, plasma, serum, saliva, cerebrospinal fluid, amniotic fluid, etc., as well as fluid collected from cell culture medium, etc.

The terms “nucleoside” and “nucleotide” are intended to include those moieties that contain not only the known purine and pyrimidine bases, but also other heterocyclic bases that have been modified. Such modifications include methylated purines or pyrimidines, acylated purines or pyrimidines, alkylated riboses or other heterocycles. In addition, the terms “nucleoside” and “nucleotide” include those moieties that contain not only conventional ribose and deoxyribose sugars, but other sugars as well. Modified nucleosides or nucleotides also include modifications on the sugar moiety, e.g., wherein one or more of the hydroxyl groups are replaced with halogen atoms or aliphatic groups, or are functionalized as ethers, amines, or the like.

The phrase “oligonucleotide bound to a surface of a solid support” refers to an oligonucleotide or mimetic thereof, e.g., peptide nucleic acid or PNA, that is immobilized on a surface of a solid substrate in a feature or spot, where the substrate can have a variety of configurations, e.g., a sheet, bead, or other structure. In certain embodiments, the collections of features of oligonucleotides employed herein are present on a surface of the same planar support, e.g., in the form of an array.

The term “array” encompasses the term “microarray” and refers to an ordered array presented for binding to nucleic acids and the like. Arrays, as described in greater detail below, are generally made up of a plurality of distinct or different features. The term “feature” is used interchangeably herein with the terms: “features,” “feature elements,” “spots,” “addressable regions,” “regions of different moieties,” “surface or substrate-immobilized elements” and “array elements,” where each feature is made up of oligonucleotides bound to a surface of a solid support, also referred to as substrate immobilized nucleic acids.

An “array,” includes any one-dimensional, two-dimensional or substantially two-dimensional (as well as a three-dimensional) arrangement of addressable regions bearing a particular chemical moiety or moieties (such as ligands, e.g., biopolymers such as polynucleotide or oligonucleotide sequences (nucleic acids), polypeptides (e.g., proteins), carbohydrates, lipids, etc.) associated with that region. In the broadest sense, the arrays of many embodiments are arrays of polymeric binding agents, where the polymeric binding agents may be any of: polypeptides, proteins, nucleic acids, polysaccharides, synthetic mimetics of such biopolymeric binding agents, etc. In many embodiments of interest, the arrays are arrays of nucleic acids, including oligonucleotides, polynucleotides, cDNAs, mRNAs, synthetic mimetics thereof, and the like. Where the arrays are arrays of nucleic acids, the nucleic acids may be covalently attached to the arrays at any point along the nucleic acid chain, but are generally attached at one of their termini (e.g. the 3′ or 5′ terminus).

In those embodiments where an array includes two more features immobilized on the same surface of a solid support, the array may be referred to as addressable. An array is “addressable” when it has multiple regions of different moieties (e.g., different polynucleotide sequences) such that a region (i.e., a “feature” or “spot” of the array) at a particular predetermined location (i.e., an “address”) on the array will detect a particular target or class of targets (although a feature may incidentally detect non-targets of that feature). Array features are typically, but need not be, separated by intervening spaces. In the case of an array, the “target” will be referenced as a moiety in a mobile phase (typically fluid), to be detected by probes (“target probes”) which are bound to the substrate at the various regions. However, either of the “target” or “probe” may be the one that is to be evaluated by the other (thus, either one could be an unknown mixture of analytes, e.g., polynucleotides, to be evaluated by binding with the other).

A “scan region” refers to a contiguous (preferably, rectangular) area in which the array spots or features of interest, as defined above, are found. The scan region is that portion of the total area illuminated from which the resulting fluorescence is detected and recorded. The term “scanning” refers to the process of reading or detecting the fluorescense signal from the scan region of an array. For the purposes of this invention, the scan region includes the entire area of the slide scanned in each pass of the lens, between the first feature of interest, and the last feature of interest, even if there are intervening areas that lack features of interest.

An “array layout” refers to one or more characteristics of the features, such as feature positioning on the substrate, one or more feature dimensions, and an indication of a moiety at a given location. “Hybridizing” and “binding”, with respect to polynucleotides, are used interchangeably.

The term “substrate” as used herein refers to a surface upon which marker molecules or probes, e.g., an array, may be adhered. Glass slides are the most common substrate for biochips, although fused silica, silicon, plastic, flexible web and other materials are also suitable.

The terms “hybridizing,” “hybridizing specifically to,” “specific hybridization,” and “selectively hybridize to,” as used herein refer to the binding, duplexing, or hybridizing of a nucleic acid molecule preferentially to a particular nucleotide sequence under stringent conditions.

The term “stringent assay conditions” as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions.

The term “sensitivity” refers to the ability of a given assay to detect a given analyte in a sample, e.g., a nucleic acid species of interest. For example, an assay has high sensitivity if it can detect a small concentration of analyte molecules in sample. Conversely, a given assay has low sensitivity if it only detects a large concentration of analyte molecules (i.e., specific solution phase nucleic acids of interest) in sample. A given assay's sensitivity is dependent on a number of parameters, including specificity of the reagents employed (e.g., types of labels, types of binding molecules, etc.), assay conditions employed, detection protocols employed, and the like. In the context of array hybridization assays, such as those of the present invention, sensitivity of a given assay may be dependent upon one or more of the nature of the surface immobilized nucleic acids, the nature of the hybridization and wash conditions, the nature of the labeling system, the nature of the detection system, etc.

In this specification and the appended claims, the singular forms “a,” “an” and “the” include plural reference unless the context clearly dictates otherwise. Unless defined otherwise, all technical and scientific terms used herein have the same meaning as commonly understood to one of ordinary skill in the art to which this invention belongs.

### Methods for Detecting Viral Nucleic Acids

In practicing embodiments, this disclosure is directed to methods and devices for detecting viral nucleic acids. Embodiments include detecting the presence of a viral vector in a host cell, detecting integration of viral nucleic acids, etc. Nucleic acid fragments obtained from host cells are amplified and then hybridized to microarrays that contains probes for genomic DNA. Hybridization of detection probes complementary to viral sequences to the same microarray allows for detection of viral nucleic acid in a host cell, i.e., determination of whether a viral nucleic acid has integrated into the host cell. The methods described herein also can determine the locus on the genome where viral integration takes place.

Methods for detecting viral nucleic acids, i.e. determining whether viral nucleic acids are present in a host cell, are described herein. In embodiments, the methods are used to detect the presence of viral DNA in a host cell, once the viral DNA has been integrated into the host genome. A target population of nucleic acid fragments (i.e. DNA or RNA fragments) from a host cell infected with a wild-type virus or a viral gene therapy vector can be generated by various methods, including methods that exploit the fusion of the viral long-terminal repeat (LTR) sequence with genomic DNA during integration. In an embodiment, integration of viral DNA is catalyzed by the viral enzyme integrase (IN), which nicks the two ends of linear viral DNA and splices the ends into the host cell genomic DNA. This produces signature DNA sequences at the junction between viral DNA and host cell genomic DNA, typically consisting of a 2 bp loss at the ends of the linear viral DNA and duplication of several base pairs of host DNA flanking the integration site.

In embodiments, PCR-based methods are used to amplify nucleic acid fragments, as described in Current Protocols in Molecular Biology, Ausubel F. M. et al., eds. 1991, the teachings of which are incorporated herein by reference. Amplification refers to a process for creating multiple copies of nucleic acids sequences and includes without limitation, methods such as inverse PCR, ligation-mediated PCR (LM-PCR), Alu-PCR, two-step PCR, etc. In other embodiments, the nucleic acid fragments generated from the host cell are sufficiently large (i.e. at least 500 bp) that no further amplification is necessary.

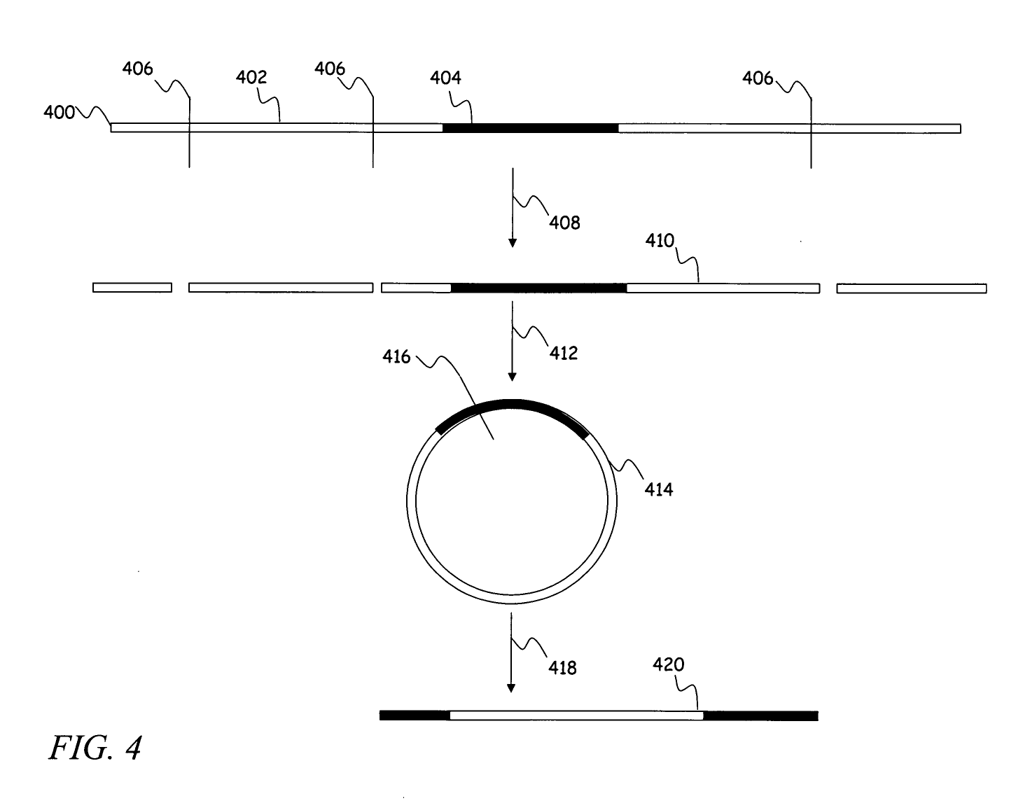

In one embodiment, nucleic acid fragments are amplified using inverse PCR. This technique provides a method for rapid in vitro amplification of nucleic acid sequence that flank a region with a known sequence. In aspects, the junction between viral LTR (either from a wild-type virus or a viral vector) and genomic DNA is circularized after digesting with a restriction enzyme and then amplified using PCR. This is a variation of the method described in Ochman et al., Genetics 120: 621-623 (1988), which is incorporated herein by reference. A simplified graphical representation of this method is shown in FIG. 4. The target DNA sequence 400 contains the integrated viral sequence 404 and unknown flanking sequences 402 with various restriction sites 406 within the flanking sequences, but not within the viral sequence. In step 408, the target DNA sequence 400 is digested with one or more restriction enzymes that cut at restriction sites 406 to produce smaller DNA fragments, along with a fragment 410 that includes the integrated viral sequence 404. The ends of fragment 410 are then self-ligated to give a circular DNA product 414. In step 418, a restriction endonuclease specific for the restriction site 416 within the viral sequence is used to linearize the fragment. The linear fragment 420 now has flanking sequences corresponding to the viral sequence flanking an unknown sequence. Fragment 420 is then amplified by PCR, using primers that are complementary to the known viral nucleic acid sequence. In embodiments, the DNA fragments produced by this method are at least 1 kb in length. Fragments as small as 200 bp may be produced, but ideally, fragments are no less than 500 bp.

In another embodiment, nucleic acid fragments are amplified using Alu-PCR. This technique provides a way to amplify nucleic acids of unknown sequence that flank a known region of the genome, but does not require ligation of the known sequence to the unknown region. This method, as applied to amplification of the human genome in the background of nonhuman genomes, was described in Nelson et al., Proc. Natl. Acad. Sci. 86: 6686-90 (1989), which is incorporated herein by reference. Briefly, the target DNA sequence containing the integrated viral sequence and unknown flanking sequences is amplified with PCR primers specific to the known viral sequence and primers specific to the Alu repeat region of the genome. This will produce two populations of PCR products: Alu-virus products and Alu-Alu products. In an aspect, the amplification of Alu-Alu products can be significantly reduced by using primers containing dUTP and treating with uracil DNA glycosylase after a few amplification cycles. The Alu-virus products are then amplified by PCR. The amplified nucleic acid fragments will include the region where the integrated viral sequence is joined to the host cell genomic sequence. In yet another embodiment, nucleic acid fragments are digested, and then either amplified by PCR methods, or left unamplified for further analysis.

In embodiments, the methods described herein are used to detect the integration of viral nucleic acids or viral gene therapy vectors into the host cell genome. Nucleic acid fragments from a host cell are isolated and amplified using techniques that exploit the fusion of the LTR sequence of the integrated viral DNA or viral vector with the host cell genomic sequence, as described above. In an embodiment, the labeled target nucleic acid fragments are hybridized to a tiling array containing probes complementary to the host cell genomic sequence. Only those nucleic acid fragments that contain viral flanking sequences will be amplified and labeled and thus available for hybridization to the tiling array.

In embodiments, the methods described herein use labeled nucleic acid sequences to detect integration of viral nucleic acids or gene therapy vectors. In an aspect, target nucleic acid fragments are labeled during amplification. Nucleic acid fragments from a host cell are digested and then amplified by PCR methods. The amplified fragments are labeled using a fluorescent dye or fluorophore, for example. The label is incorporated into the target nucleotide fragment. As a result, the target nucleotide sequences, when hybridized to a microarray, can be detected directly, without the use of a secondary detection probe. In another aspect, unlabeled target nucleic acid fragments are first hybridized to a set of secondary oligonucleotide probes with sequences complementary to the viral nucleic acid of interest, i,e., detection probes. These probes are labeled with a tag, such a fluorescent marker of fluorophore. The target fragments and the labeled detection probes are then hybridized to a tiling array containing probes complementary to the host cell genome. In yet another aspect, the target nucleic acid fragments are first hybridized to a tiling array, and then secondarily hybridized to the detection probes. In alternate embodiments, the target fragments are hybridized to the array and simultaneously hybridized to the detection probe, in a single hybridization reaction. In embodiments, the target nucleic acid fragments can be crosslinked to the tiling array after hybridization. In alternate embodiments, crosslinking is not used, with the binding of the nucleic acid fragments to the array or detection probes controlled by the stringency of the hybridization.

In embodiments, the detection probes are oligonucleotides with sequences complementary to the integrated provirus. On hybridization, only those nucleic acid fragments that include flanking sequences derived from the integrated provirus will bind. The detection probe is labeled with a fluorescent dye, or a fluorophore (such as Cy3, for example). Using a microarray scanner, the fluorescently labeled probes are detected. Only those regions of the array that have viral flanking sequences light up. Because each locus of the array corresponds to a known region of the genome, the location of the detected fragments provides information on the locus of viral integration in the genome. This method can also be used to detect multiple integration sites with a host cell population. The relative fluorescent intensity of different sites gives information as to the relative proportion of host cells within the population that have an integration. The method can also be used to determine if a tandem integration has occurred at a given site on the genome. In another embodiment, the amplified target nucleic acid fragments and the detection probes are differentially labeled with fluorescent dyes, or a fluorophore (such as Cy3 and Cy5, for example). This method can ensure that all regions were properly amplified, and that no integration sites were missed because of improper amplification or hybridization.

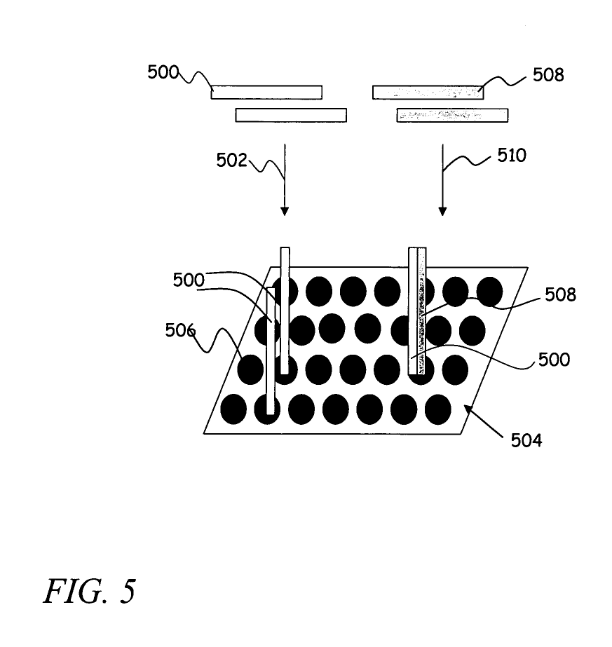

An embodiment of this method is illustrated in FIG. 5. Nucleic acid fragments 500 isolated from the host cell (some of which contain viral nucleic acid flanking sequences) are hybridized in step 502 to a tiling array 504, which contains oligonucleotide probes 506 with sequences complementary to the sequence of the host cell genome. In step 510, the fragments 500 are further hybridized with fluorescently labeled detection probes 508. These probes are complementary to viral nucleic acids and will bind with nucleic acid fragments 500 that contain viral flanking sequences. Because of the fluorescent tag, the particular locus of the array where this binding takes place will light up (i.e. a fluorescent signal will be seen). The presence of the fluorescent signal indicates that a virus has been integrated into the host genome. Furthermore, as each locus of the array represents a particular locus of the genome, the location of the fluorescent signal on the array indicates the site on the genome where the virus or viral vector has integrated.

The present methods are for detecting and analyzing a wide variety of viruses and viral vectors that can integrate into a host cell genome. Many viruses, including retroviruses, adeno-associated viruses, DNA tumor viruses, and viral vectors designed for use in gene therapy can undergo integration. Viral DNA (or the provirus) is integrated into the host genome by the action of the integrase (IN) enzyme. This integration event provides a tag that marks a particular time in evolution and can be used as a way to study speciation, divergence, etc. The integration event can also be used to determine the mode of action of antiviral drugs, such as integrase inhibitors, for example.

The methods described herein can be used to analyze the mutagenic activity of viruses, especially retroviruses and adeno-associated viruses. For example, the integration of proviral DNA or of a viral gene therapy vector into the host genome causes gross alterations in the genome. Such alterations can have deleterious effects such as activation of an oncogene, or knocking out a tumor suppressor gene, for example. The methods described herein can therefore be used to determine the location of the proviral integration and thereby identify new oncogenes. The methods described herein provide an effective tool for detecting genetic alterations and the effect of such alterations on normal cell growth and metabolism.

### Arrays Used for Detection of Viral Nucleic Acids

The presence of viral nucleic acids in the host cell genome is detected by probing the nucleic acid (or DNA) fragments with oligonucleotide sequences complementary to viral nucleic acid (or DNA) sequences. The isolated nucleic acid fragments, amplified by any of the methods described, are hybridized to oligonucleotide probes immobilized on a DNA array, or microarray. In an aspect, a microarray contains spots or features corresponding to host cell genomic DNA sequences. In another aspect, the array includes spots or features corresponding to viral nucleic acid sequences. In embodiments, the DNA array is a tiling array, i.e. a type of microarray where probes are not designed to target known genes or promoters, but are simply laid down at regular intervals along the length of the genome. Tiling arrays include overlapping nucleotides designed to blanket the entire genome, or an entire genomic region of interest. The interval spacing (or resolution of the array) can be varied according to the application for which the tiling array is used. Typically, the interval spacing can range from about 5 bp to about 500 bp, for a tiling array containing 10 chromosomes, for example. Tiling arrays of the type described herein are commercially available.

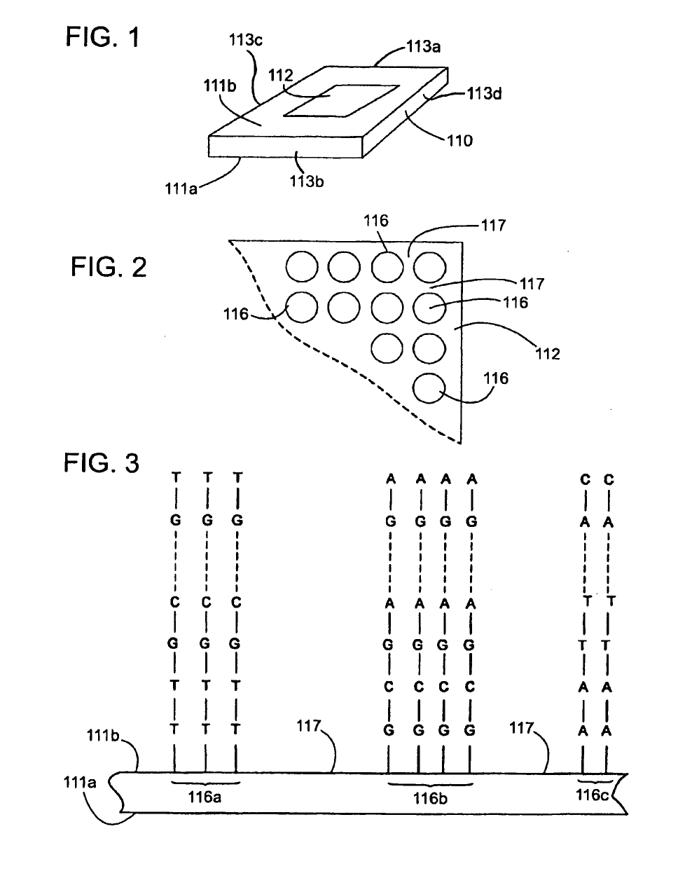

The isolated and/or amplified nucleic acid fragments obtained from the host cell are probed with oligonucleotide sequences corresponding to genomic DNA and viral DNA, using a number of different techniques. In one embodiment, complementary sequences are immobilized onto a glass slide or microchip to form a DNA microarray. An exemplary array is shown in FIGS. 1-3. The array shown in this representative embodiment includes a contiguous planar substrate 110 carrying an array 112 disposed on a rear surface 111b of substrate 110. It will be appreciated though, that more than one array (any of which are the same or different) may be present on rear surface 111b, with or without spacing between such arrays. That is, any given substrate may carry one, two, four or more arrays disposed on a front surface of the substrate and depending on the use of the array, any or all of the arrays may be the same or different from one another and each may contain multiple spots or features. The one or more arrays 112 usually cover only a portion of the rear surface 111b, with regions of the rear surface 111b adjacent the opposed sides 113c, 113d and leading end 113a and trailing end 113b of slide 110, not being covered by any array 112. A front surface 111a of the slide 110 does not carry any arrays 112. Each array 112 can be designed for testing against any type of sample, whether a trial sample, reference sample, a combination of them, or a known mixture of biopolymers such as polynucleotides. Substrate 110 may be of any shape.

As mentioned above, array 112 contains multiple spots or features 116 of biopolymers, e.g., in the form of polynucleotides. All of the features 116 may be different, or some or all could be the same. The interfeature areas 117 could be of various sizes and configurations. Each feature carries a predetermined biopolymer such as a predetermined polynucleotide (which includes the possibility of mixtures of polynucleotides). It will be understood that there may be a linker molecule (not shown) of any known types between the rear surface 111b and the first nucleotide.

Substrate 110 may carry on front surface 111a, an identification code, e.g., in the form of bar code (not shown) or the like printed on a substrate in the form of a paper label attached by adhesive or any convenient means. The identification code contains information relating to array 112, where such information may include, but is not limited to, an identification of array 112, i.e., layout information relating to the array(s), etc.

The DNA arrays described herein are arrays of nucleic acids, including oligonucleotides, polynucleotides, DNAs, RNAs, synthetic mimetics thereof, and the like. Specifically, the arrays contain spots or features in the form of oligonucleotides corresponding to specific probe sequences. The subject arrays include at least two distinct nucleic acids that differ by monomeric sequence immobilized on, e.g., covalently to, different and known locations on the substrate surface. In an embodiment, the arrays contain spots corresponding to genomic DNA sequences, as well as proviral DNA sequences. In certain embodiments, each distinct nucleic acid sequence of the array is typically present as a composition of multiple copies of the polymer on the substrate surface, e.g., as a spot on the surface of the substrate. The number of distinct nucleic acid or oligonucleotide sequences, or spots or similar structures present on the array may vary, but is generally at least 2, usually at least 5 and more usually at least 10, where the number of different spots on the array may be as a high as 50, 100, 500, 1000, 10,000, 100,000 or higher, depending on the intended use of the array. The spots of distinct oligonucleotide sequences present on the array surface are generally present as a pattern, where the pattern may be in the form of organized rows and columns of spots, e.g., a grid of spots, across the substrate surface, a series of curvilinear rows across the substrate surface, e.g., a series of concentric circles or semi-circles of spots, and the like. The density of spots present on the array surface may vary, but will generally be at least about 10 and usually at least about 100 spots/cm², where the density may be as high as 10⁶ or higher, but will generally not exceed about 10⁵ spots/cm². In other embodiments, the oligonucleotide sequences are not arranged in the form of distinct spots, but may be positioned on the surface such that there is substantially no space separating one polymer sequence/feature from another.

Arrays can be fabricated using drop deposition from pulsejets of either polynucleotide precursor units (such as monomers) in the case of in situ fabrication, or the previously obtained polynucleotide. In an embodiment, the arrays are fabricated using oligonucleotides with sequences complementary to host cell genomic DNA. In another embodiment, the arrays are fabricated using oligonucleotides with sequences complementary to viral nucleic acids. In yet another embodiment, the arrays are fabricated as tiling arrays, with oligonucleotide probes simply laid down at regular intervals along the length of the genome or along the length of a genomic region of interest. Methods for array fabrication are described in detail in, for example, U.S. Pat. No. 6,242,266, U.S. Pat. No. 6,232,072, U.S. Pat. No. 6,180,351, U.S. Pat. No. 6,171,797, U.S. Pat. No. 6,323,043, U.S. patent application Ser. No. 09/302,898 filed Apr. 30, 1999 by Caren et al., and the references cited therein. These references are incorporated herein by reference. Other drop deposition methods can be used for fabrication.

In embodiments, the methods described herein use a tiling array where the resolution depends on the size of the fragments generated during the isolation or amplification stage. For example, a tiling array with a resolution of 500 bp is used when the fragments produced by amplification are about 200 bp to about 500 bp in length. A typical tiling array, as used in the methods herein, uses 60-mer nucleotide sequences, wherein each 60-mer is a sequence beginning about 500 bp from the previous sequence along the length of the genome. Furthermore, each 60-mer is spaced apart from the adjacent 60-mers by a regular interval determined by the length of the DNA fragments isolated from the host cell. In some embodiments, the arrays use 25-mer oligonucleotides sequences, and in other embodiments, the arrays contain 200-mer oligonucleotide sequences spotted onto the array.

In the methods described herein, the presence of an integrated viral nucleic acid is detected by hybridization of isolated DNA fragments to a microarray. The hybridization step involves contacting the tiling array with the target nucleic acid fragments from the host cell. Nucleic acid fragments with sequences complementary to the oligonucleotides on the array will bind. The array is then washed to remove non-specifically bound nucleic acids, and then crosslinked to more strongly bind nucleic acids already bound to the array. Various methods can be used for crosslinking including, but not limited to, UV light. In the alternative, the crosslinking step may be omitted, and the target nucleic acid fragments and the detection probe can be hybridized to the microarray at the same time. In this case, effective binding of the target nucleic acids to the microarray or to the detection probe requires careful control of the stringency of hybridization.

In embodiments, the DNA fragments are hybridized to the microarray under stringent assay conditions. Stringent assay conditions as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions. A stringent hybridization and stringent hybridization wash conditions in the context of nucleic acid hybridization (e.g., as in array, Southern or Northern hybridizations) are sequence dependent, and are different under different experimental parameters.

Stringent hybridization conditions that can be used to identify nucleic acids can include, e.g., hybridization in a buffer comprising 50% formamide, 5×SSC, and 1% SDS at 42° C., or hybridization in a buffer comprising 5×SSC and 1% SDS at 65° C., both with a wash of 0.2×SSC and 0.1% SDS at 65° C. Exemplary stringent hybridization conditions can also include a hybridization in a buffer of 40% formamide, 1 M NaCl, and 1% SDS at 37° C., and a wash in 1×SSC at 45° C. Alternatively, hybridization to filter-bound DNA in 0.5 M NaHPO₄, 7% sodium dodecyl sulfate (SDS), 1 mM EDTA at 65° C., and washing in 0.1×SSC/0.1% SDS at 68° C. can be employed. Yet additional stringent hybridization conditions include hybridization at 60° C. or higher and 3×SSC (450 mM sodium chloride/45 mM sodium citrate) or incubation at 42° C. in a solution containing 30% formamide, 1M NaCl, 0.5% sodium sarcosine, 50 mM MES, pH 6.5. Those of ordinary skill will readily recognize that alternative but comparable hybridization and wash conditions can be utilized to provide conditions of similar stringency. For example, in the methods described herein, hybridization is accomplished using a buffer composition as described in U.S. Patent Publication No. 20030013092. The buffer composition comprises a non-chelating buffering agent with a pH in the range of about 6.4 to 7.5, and a monovalent cation with concentration in the range of 0.01M to about 2.0M. Optionally, relatively lower concentrations of a chelating agent and a nonionic surfactant are included. For hybridization, the target nucleic acids are incubated with the microarray in the buffer composition at temperatures between about 55° C. and about 70° C.

In certain embodiments, the stringency of the wash conditions sets forth the conditions that determine whether a nucleic acid is specifically hybridized to a surface bound nucleic acid. Wash conditions used to identify nucleic acids may include, e.g.: a salt concentration of about 0.02 molar at pH 7 and a temperature of at least about 50° C. or about 55° C. to about 60° C.; or, a salt concentration of about 0.15 M NaCl at 72° C. for about 15 minutes; or, a salt concentration of about 0.2×SSC at a temperature of at least about 50° C. or about 55° C. to about 60° C. for about 15 to about 20 minutes; or, the hybridization complex is washed twice with a solution with a salt concentration of about 2×SSC containing 0.1% SDS at room temperature for 15 minutes and then washed twice by 0.1×SSC containing 0.1% SDS at 68° C. for 15 minutes; or, equivalent conditions. Stringent conditions for washing can also be, e.g., 0.2×SSC/0.1% SDS at 42° C.

Stringent assay conditions are hybridization conditions that are at least as stringent as the above representative conditions, where a given set of conditions are considered to be at least as stringent if substantially no additional binding complexes that lack sufficient complementarity to provide for the desired specificity are produced in the given set of conditions as compared to the above specific conditions, where by “substantially no additional” is meant less than about 5-fold more, typically less than about 3-fold more. Other stringent hybridization conditions are known in the art and may also be employed, as appropriate.

### Kits for Detection of Viral Nucleic Acids

In embodiments, the methods described herein can be used in kits for the identification or detection of viral nucleic acids that have become integrated into the host cell genome. The kits contain at least one suitably packaged microarray with spots corresponding to probes for host cell genomic DNA or viral or viral vector DNA. In embodiments, the microarray of the kit can be a tiling array containing spots or features laid down at regular intervals along the length of the genome, or a genomic region of interest. In embodiments, the kits described herein contain oligonucleotide probes with sequences complementary to the integrated provirus, i.e. detection probes. In embodiments, the kits described herein contain reagents required for amplification of nucleic acid fragments. These reagents include, for example, PCR primers, restriction enzymes or endonucleases, such as endonucleases capable of cutting within a proviral sequence, etc. The kits may also contain instructions providing information on use of the microarray to detect the presence and/or integration of viral nucleic acids. In embodiments, the kits also contain fluorophores for differential labeling of amplified DNA, reagents for amplifying DNA fragments using PCR, etc.

The various embodiments described above are provided by way of illustration only and should not be construed to limit the invention. Those skilled in the art will readily recognize various modifications and changes that may be made to the present invention without following the example embodiments and applications illustrated and described herein, and without departing from the true spirit and scope of the present invention without following the example embodiments and applications illustrated and described herein, and without departing from the true spirit and scope of the present invention, which is set forth in the following claims.

1. A method for detecting integration of a viral nucleic acid of interest into a host cell genome, comprising the steps of:
generating a target population of nucleic acid fragments from the host cell;
hybridizing the target population of nucleic acid fragments to a microarray; and
scanning the microarray to detect the target population of nucleic acid fragments,
wherein the location of the integrated viral nucleic acid on the microarray further indicates a genomic integration site.

2. The method of claim 1, wherein generating the target population of nucleic acid fragments comprises amplification by inverse PCR.

3. The method of claim 1, wherein generating the target population of nucleic acid fragments comprises amplification by Alu-PCR.

4. The method of claim 1, wherein hybridizing the target population of nucleic acid fragments on the microarray comprises:
hybridizing the target population of nucleic acid to detection probes with sequences complementary to the viral nucleic acid of interest; and
detecting the detection probe to determine the presence of an integrated viral nucleic acid.

5. The method of claim 1, wherein hybridizing the target population of nucleic acid fragments on the microarray comprises detecting the target population of nucleic acid fragments directly, without the use of a detection probe.

6. The method of claim 4, wherein hybridization of the target population of nucleic acid fragments to the microarray, and hybridization of the target population of nucleic acid fragments to the detection probes occur simultaneously.

7. The method of claim 1, wherein hybridizing the target population of nucleic acid fragments to a microarray comprises:
contacting the microarray with the target population of nucleic acid fragments to bind nucleic acid fragments to microarray; and
washing the microarray to remove nucleic acid fragments not bound to the microarray.

8. The method of claim 7, wherein hybridization further comprises crosslinking the microarray to more strongly bind nucleic acid fragments already bound to the microarray.

9. The method of claim 1, wherein the viral nucleic acid of interest comprises a viral vector used for gene therapy.

10. The method of claim 1, wherein the host cell is a mammalian cell.

11. The method of claim 1, wherein the target nucleic acid fragments are labled with a fluorophore, or a fluorescent dye.

12. The method of claim 1, wherein the detection probes are labeled with a fluorophore, or a fluorescent dye.

13. The method of claim 1, wherein the target nucleic acid fragments and the detection probes are differentially labeled, further comprising labeling each with a different fluorophore, or a different fluorescent dye.

14. The method of claim 1, wherein the microarray is a tiling array.

15. A nucleotide array, comprising a plurality of oligonucleotides immobilized on a substrate, wherein the plurality comprises polynucleotides with sequences complementary to viral DNA or host cell genomic DNA, and wherein the plurality of oligonucleotides are placed at distinct loci, each locus being separated by the length of target nucleic acid fragments being analyzed using the array.

16. The array of claim 15, wherein the oligonucleotides at each locus are at least 60 bp in length.

17. A kit for detecting the integration of a viral nucleic acid of interest into a host cell genome, comprising:
at least one microarray containing oligonucleotides with sequences complementary to host cell genomic DNA;
at least one oligonucleotide probe with sequence complementary to the viral nucleic acid of interest; and
instructions for the use of the kit to detect the integration of a viral nucleic acid into the host cell genome.

18. The kit of claim 17, further comprising:
a restriction endonuclease capable of cutting within various sequences of the genome;
a restriction endonuclease capable of specifically cutting within the known sequence of a viral nucleic acid; and
primers for PCR amplification.

19. The kit of claim 17, wherein the microarray comprises a nucleotide array containing oligonucleotides at least 60 bp in length placed at distinct loci on the array, each locus being separated by the length of target nucleic acid fragments being analyzed using the kit.
